(19) United States
(12) Patent Application Publication
(10) Pub. No.: US 2019/0120048 A1
(43) Pub. Date: Apr. 25, 2019

(54) USING FLUIDIC DEVICES TO ESTIMATE WATER CUT IN PRODUCTION FLUIDS

(71) Applicant: Halliburton Energy Services, Inc.

(72) Inventors: Maxime PM COFFIN, Frisco, TX (US); Michael Linley FRIPP, Carrollton, TX (US); Georgina Corona CORTES, Plano, TX (US); Andrew David PENNO, Singapore (SG)

(21) Appl. No.: 15/551,897

(22) PCT Filed: Sep. 27, 2016
(86) PCT No.: PCT/US2016/053897

§ 371 Date: Aug. 17, 2017

(51) Int. Cl. E21B 49/08; E21B 47/06; E21B 47/10; G01N 33/28

(52) U.S. Cl. CPC E21B 49/08; E21B 47/06; E21B 47/065; E21B 43/08; G01N 33/2823; G01N 33/2847; E21B 2049/085; E21B 47/101

(57) ABSTRACT

A method includes drawing a fluid into a flow control assembly coupled to a completion string positioned within a wellbore, the flow control assembly including a first fluidic device and a second fluidic device, where the first and second fluidic devices exhibit different flow characteristics. A flow condition of the fluid circulating through the first and second fluidic devices is measured with a plurality of fluid sensors, and a water cut of the fluid is estimated based on the flow condition measured by the plurality of fluid sensors.

### BACKGROUND

In hydrocarbon production wells, it is often beneficial to regulate the flow of formation fluids from a subterranean formation into a wellbore penetrating the same. A variety of reasons or purposes can necessitate such regulation including, for example, prevention of water and/or gas coning, minimizing water and/or gas production, minimizing sand production, maximizing oil production, balancing production from various subterranean zones, equalizing pressure among various subterranean zones, and/or the like.

A number of fluidic devices or modules are available for regulating the flow of formation fluids. Some of these devices are non-discriminating for different types of formation fluids and can simply function as a “gatekeeper” for regulating access to the interior of a wellbore pipe, such as a well string. Such gatekeeper devices can be simple on/off valves or they can be metered to regulate fluid flow over a continuum of flow rates. Other types of devices for regulating the flow of formation fluids can achieve at least some degree of discrimination between different types of formation fluids. Such devices can include, for example, tubular flow restrictors, nozzle-type flow restrictors, autonomous inflow control devices (AICD), non-autonomous inflow control devices, ports, tortuous paths, combinations thereof, and the like.

While regulating the flow of formation fluids, it is advantageous to know what proportion of certain fluids (e.g., hydrocarbons) are being produced as opposed to other fluids (e.g., water). When it is determined that a production interval is producing more of one type of fluid than other fluids, a well operator may then decide to reduce or cease production from that production interval, which will result in more efficient production operations for the well.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a schematic diagram of an exemplary well system that may employ one or more of the principles of the present disclosure.

FIG. 2 is a partial cross-sectional view of successive axial sections of an example flow control assembly.

FIG. 3A is a schematic view of an example embodiment of the flow control section of FIG. 2.

FIG. 3B is a schematic view of another example embodiment of the flow control section of FIG. 2.

FIGS. 4A-4H are cross-sectional side views of a variety of example fluidic devices that may be employed in accordance with the principles of the present disclosure.

FIG. 5 is a schematic diagram of an example fluid circuit.

FIG. 6 is a plot depicting test results for two example fluidic devices that help provide operational data for the fluidic devices.

FIG. 7 is a plot showing test results for the two example fluidic devices of FIG. 6 in determining water cut.

FIG. 8 is a schematic diagram of another example fluid circuit used to help determine water cut.

FIG. 9 is a schematic diagram of another example fluid circuit used to help estimate water cut.

### DETAILED DESCRIPTION

The present disclosure relates to downhole fluid flow regulation and, more particularly, to estimating water cut (or alternatively oil fraction) in a producing interval using fluidic devices and fluid sensors.

The embodiments discussed herein describe the use of a plurality of fluidic devices arranged in a flow control assembly of a downhole completion to help estimate the water cut in a subterranean production fluid. The fluidic devices exhibit different but known flow resistances to fluids having known fluid properties (e.g., viscosity, density, etc.). The water cut can be estimated by circulating the fluid through the fluidic devices and measuring a flow condition of the fluid with a plurality of fluid sensors. The water cut of the fluid may then be estimated based on the flow condition measured by the plurality of fluid sensors. If the water cut is estimated to exceed a predetermined limit, a well operator may be able to choke or stop flow of the fluid from that location. The principles of the present disclosure may also be employed in estimating the gas cut in a subterranean production fluid.

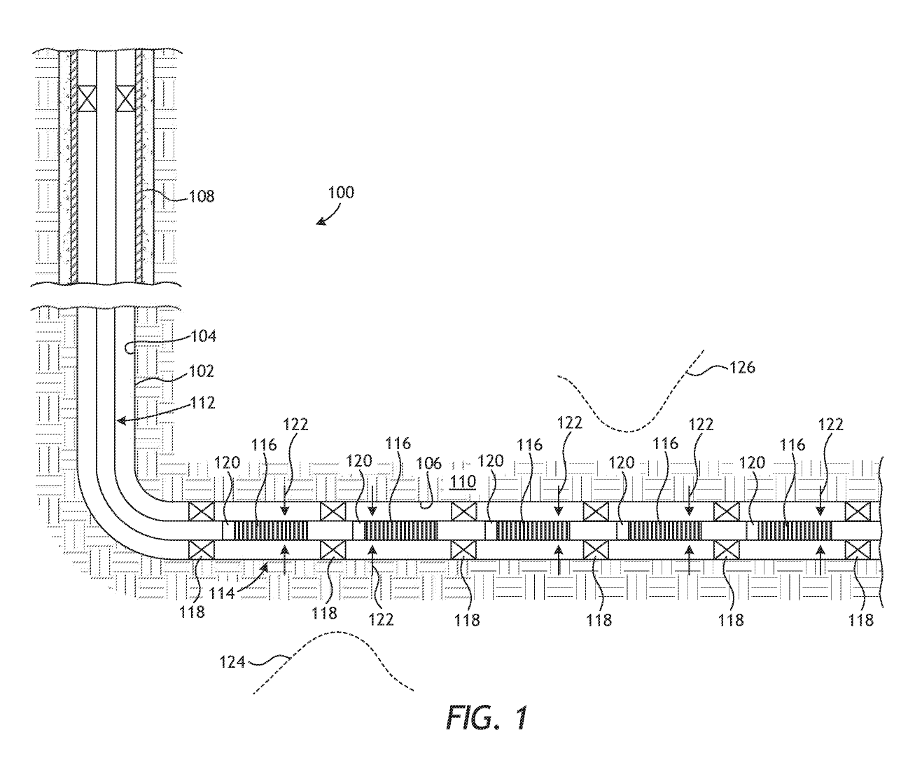

FIG. 1 is a schematic diagram of an exemplary well system 100 that may employ one or more of the principles of the present disclosure, according to one or more embodiments. As depicted, the well system 100 includes a wellbore 102 that extends through various earth strata and has a substantially vertical section 104 that transitions into a substantially horizontal section 106. A portion of the vertical section 104 may have a string of casing 108 cemented therein, and the horizontal section 106 may extend through a hydrocarbon bearing subterranean formation 110. In some embodiments, the horizontal section 106 may be uncompleted and otherwise characterized as an “open hole” section of the wellbore 102. In other embodiments, however, the casing 108 may extend into the horizontal section 106, without departing from the scope of the disclosure.

A string of production tubing 112 may be positioned within the wellbore 102 and extend from a surface location (not shown), such as the Earth's surface. The production tubing 112 provides a conduit for fluids extracted from the formation 110 to travel to the surface location for production. A completion string 114 may be coupled to or otherwise form part of the lower end of the production tubing 112 and arranged within the horizontal section 106. The completion string 114 divides the wellbore 102 into various production intervals adjacent the subterranean formation 110. To accomplish this, as depicted, the completion string 114 may include a plurality of flow control assemblies 116 axially offset from each other along portions of the production tubing 112. Each flow control assembly 116 may be positioned between a pair of wellbore packers 118 that provides a fluid seal between the completion string 114 and the inner wall of the wellbore 102, and thereby defining discrete production intervals. One or more of the flow control assemblies 116 may further include at least one fluidic device 120 used to convey or otherwise regulate the flow of fluids 122 (i.e., a production fluid stream) into the completion string 114 and, therefore, into the production tubing 112.

In operation, each flow control assembly 116 serves the primary function of filtering particulate matter out of the fluids 122 originating from the formation 110 such that particulates and other fines are not produced to the surface. The fluidic devices 120 then operate to regulate the flow of the fluids 122 into the completion string 114. Regulating the flow of fluids 122 in each production interval may be advantageous in preventing water coning 124 or gas coning 126 in the subterranean formation 110. Other uses for flow regulation of the fluids 122 include, but are not limited to, balancing production from multiple production intervals, minimizing production of undesired fluids, maximizing production of desired fluids, etc.

In the illustrated embodiment, each flow control assembly 116 includes one or more sand screens that serve as a filter medium to filter the incoming fluids 122. The sand screens, however, may be replaced with any other type of filter medium, such as a slotted liner or the like, without departing from the scope of the disclosure. In yet other embodiments, the filter medium may be omitted from one or more of the flow control assemblies 116 and the incoming fluids 122 may instead be conveyed directly to the fluidic devices 120 without filtration. Accordingly, use of the sand screens in FIG. 1 is for illustrative purposes only and should not be considered limiting to the present disclosure.

It should be noted that even though FIG. 1 depicts the flow control assemblies 116 as being arranged in an open hole portion of the wellbore 102, embodiments are contemplated herein where one or more of the flow control assemblies 116 is arranged within cased portions of the wellbore 102. Also, even though FIG. 1 depicts a single flow control assembly 116 arranged in each production interval, any number of flow control assemblies 116 may be deployed within a particular production interval without departing from the scope of the disclosure. In addition, even though FIG. 1 depicts multiple production intervals separated by the packers 118, any number of production intervals with a corresponding number of packers 118 may be used. In other embodiments, the packers 118 may be entirely omitted from the completion interval, without departing from the scope of the disclosure.

Furthermore, while FIG. 1 depicts the flow control assemblies 116 as being arranged in the horizontal section 106 of the wellbore 102, the flow control assemblies 116 are equally well suited for use in the vertical section 104 or portions of the wellbore 102 that are deviated, slanted, multilateral, or any combination thereof. Moreover, while FIG. 1 generally depicts a land-based drilling assembly, those skilled in the art will readily recognize that the principles described herein are equally applicable to subsea operations that employ floating or sea-based platforms and rigs, without departing from the scope of the disclosure. The use of directional terms such as above, below, upper, lower, upward, downward, left, right, uphole, downhole and the like are used in relation to the illustrative embodiments as they are depicted in the figures, the upward direction being toward the top of the corresponding figure and the downward direction being toward the bottom of the corresponding figure, the uphole direction being toward the surface of the well and the downhole direction being toward the toe of the well.

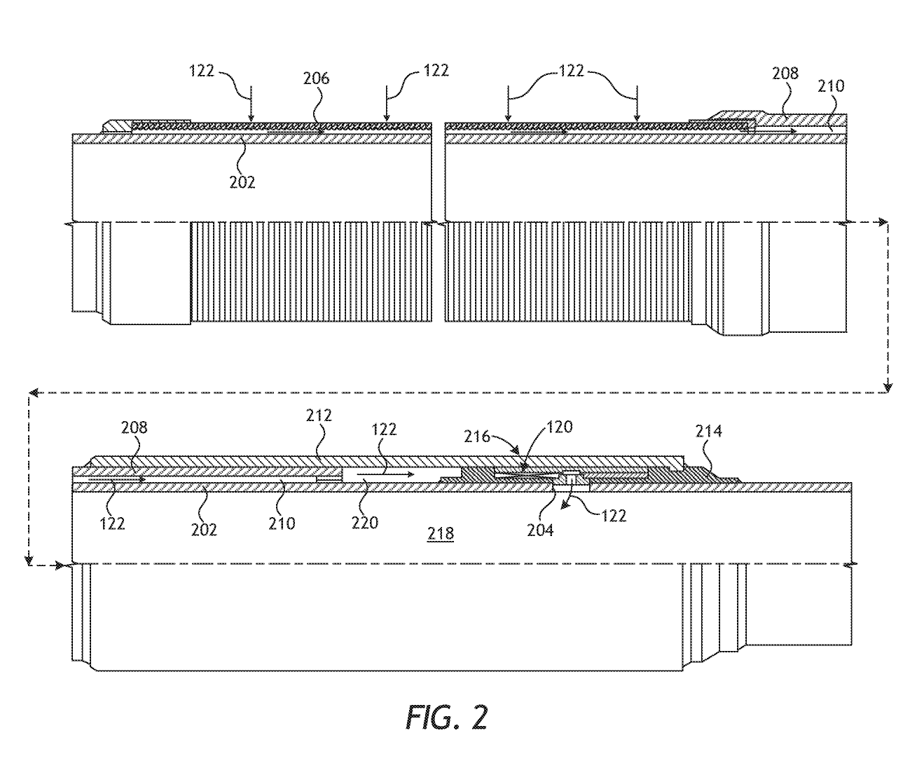

FIG. 2 is a partial cross-sectional view of successive axial sections of an example flow control assembly 116, according to one or more embodiments. The flow control assembly 116 may be any of the flow control assemblies 116 shown in FIG. 1. As illustrated, the flow control assembly 116 includes a base pipe 202 that defines one or more production ports 204. The base pipe 202 forms part of the completion string 114 (FIG. 1) and otherwise fluidly communicates with the production tubing 112 (FIG. 1). A filter medium 206 is positioned around (about) an uphole portion of the base pipe 202. As illustrated, the filter medium 206 comprises a screen element, such as a wire wrap screen, a woven wire mesh screen, a prepacked screen or the like, but could alternatively comprise a slotted pipe. The filter medium 206 is designed to allow fluids to flow therethrough but prevent particulate matter of a predetermined size from flowing therethrough. As indicated above, however, the filter medium 206 may alternatively be omitted from the flow control assembly 116.

Positioned downhole of the filter medium 206 is a screen interface housing 208 that forms an annulus 210 jointly with the base pipe 202. A flow control shroud 212 is secured to the downhole end of the screen interface housing 208. At its downhole end, the flow control shroud 212 is securably connected to a support assembly 214, which is secured to base pipe 202. The various connections of the components of the flow control assembly 116 may be made in any suitable fashion including welding, threading, and the like, as well as through the use of various mechanical fasteners, such as bolts, screws, pins, snap rings, etc.

Positioned between the support assembly 214 and the flow control shroud 212 are a plurality of fluidic devices, generally depicted at reference numeral 120. The fluidic devices 120 may be alternately referred to as “fluidic modules,” “fluidic components,” and “fluid diodes.” In some embodiments, the fluidic devices 120 may be configured to convey incoming fluids into the base pipe 202 via the flow port(s) 204. In other embodiments, however, the one or more of the fluidic devices 120 may be configured to regulate or control the flow of incoming fluids. In such embodiments, the fluidic devices 120 may comprise, for example, inflow control devices (ICD) or autonomous inflow control devices (AICD). An ICD is designed to exhibit a viscosity dependent fluid flow resistance in the form of a positive flowrate response to decreasing fluid viscosity. In contrast, an AICD is designed to exhibit a viscosity dependent fluid flow resistance in the form of a negative flowrate response to decreasing fluid viscosity. Flow changes through the ICD and/or the AICD can be a function of density and flow rate, in addition to viscosity. In some embodiments, the same ICD or AICD may exhibit a positive and a negative flowrate response depending on the flow regime. More particularly, a given ICD or AICD may exhibit a negative flow rate response for one combination of viscosity, flow rate, and density, but may exhibit a positive flow rate response for a different combination of viscosity, flow rate, and density, without departing from the scope of the disclosure.

The fluidic devices 120 may be positioned about the circumference of the base pipe 202 within a flow control section 216 in a variety of configurations. In some embodiments, for example, two or more of the fluidic devices 120 may be arranged in parallel within the flow control section 216. In other embodiments, or in addition thereto, two or more of the fluidic devices 120 may be arranged in series within the flow control section 216, without departing from the scope of the disclosure. Moreover, the fluidic devices 120 may be circumferentially distributed at uniform or non-uniform intervals about the periphery of the base pipe 202.

The fluidic devices 120 are fluidly coupled to and otherwise in fluid communication with the production port(s) 204. Accordingly, the fluidic devices 120 operate to control the flow of fluids 122 into a central flow passage 218 defined by the base pipe 202 via the production port(s) 204. In example operation, and during the production phase of well operations, the fluid 122 is drawn into the flow control assembly 116 from a surrounding formation (i.e., the formation 110 of FIG. 1). After being filtered by the filter medium 206, if present, the fluid 122 flows into the annulus 210, which communicates with an annular region 220 defined between the base pipe 202 and the flow control shroud 212. The fluid 122 then circulates into the fluid circuit provided by the flow control section 216 and otherwise to the inlets of the fluidic devices 120 where desired flow regulation occurs depending upon the composition of the fluid 122. The fluidic devices 120 then expel the fluid 122 toward the production port(s) 204 to be discharged into the central flow passage 218 for production to the well surface.

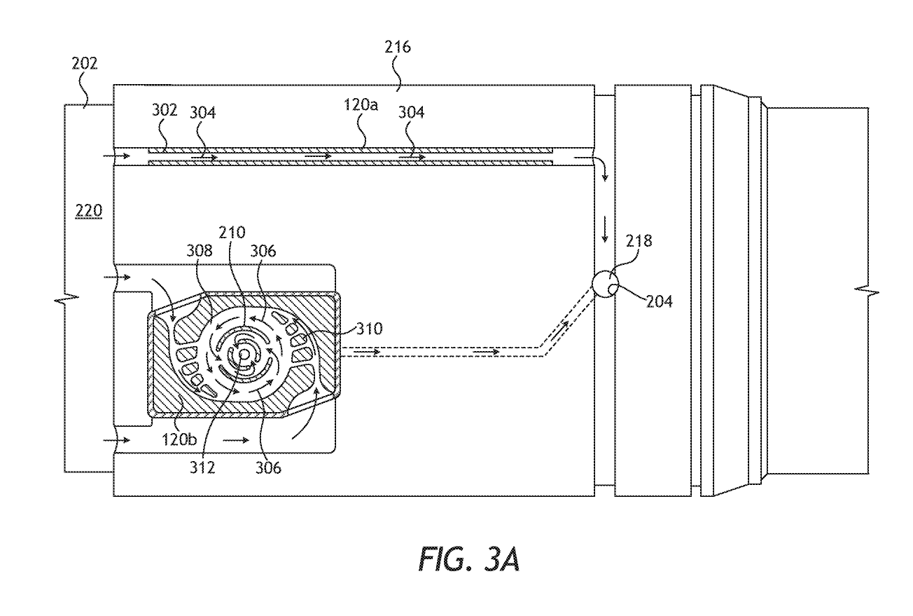

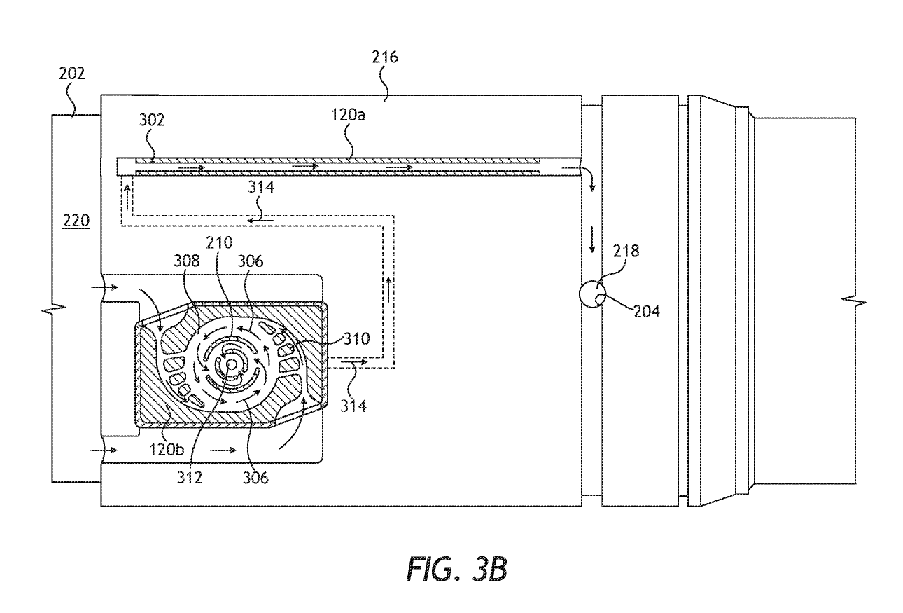

FIG. 3A is a schematic view of an example embodiment of the flow control section 216 of FIG. 2, according to one or more embodiments. The flow control shroud 212 (FIG. 2) has been removed in FIG. 3A to enable viewing of the fluidic devices included in the fluid circuit of the flow control section 216. The fluidic devices are depicted as a first fluidic device 120a and a second fluidic device 120b arranged in parallel and in fluid communication with the production port(s) 204 (only one shown).

The first fluidic device 120a is depicted as an inflow control device (ICD) that provides resistance to fluid flow therethrough, as indicated by arrows 304. More specifically, the first fluidic device 120a is depicted in the form of a flow tube 302. In the case of a relatively high viscosity fluid composition containing predominately oil, flow through the first fluidic device 120a encounters relatively high resistance. On the other hand, in the case of a relatively low viscosity fluid composition containing predominately water, flow through the first fluidic device 120a encounters relatively low resistance. The first fluidic device 120a thus has viscosity dependent fluid flow resistance and in particular, a positive flowrate response to decreasing fluid viscosity.

The second fluidic device 120b is depicted as an autonomous inflow control device (AICD) that also provides resistance to fluid flow therethrough, as indicated by arrows 306. More specifically, the second fluidic device 120b is depicted in the form of a fluid diode having a vortex chamber 308 in which one or more fluid guides 310 are provided. The second fluidic device 120b is sometimes referred to as a “vortex chamber diode.” In the case of a relatively high viscosity fluid composition containing predominately oil, flow through the second fluidic device 120b may progress relatively unimpeded. On the other hand, in the case of a relatively low viscosity fluid composition containing predominately water, the fluids entering the vortex chamber 308 will travel primarily in a tangentially direction and will spiral around the vortex chamber 308 with the aid of the fluid guides 310 before eventually exiting through a centrally-located outlet 312. In other embodiments, the fluid circulating through the vortex chamber 308 may be rotated and translated on a helical path and still generally function the same.

Fluid spiraling around the vortex chamber 308 will suffer from frictional losses. Further, the tangential velocity produces centrifugal force that impedes radial flow. Consequently, spiraling fluids passing through the second fluidic device 120b encounter significant resistance. The more circuitous the flow path taken by the relatively low viscosity fluid composition, the greater the amount of energy consumed. This can be compared with the more direct flow path taken by the relatively high viscosity fluid composition in which a lower amount of energy consumed. In this example, if oil and water are being circulated, the second fluidic device 120b will provide low resistance to fluid flow when the fluid composition has a relatively high ratio of oil-to-water, and will provide progressively greater resistance as the ratio of oil-to-water decreases. The second fluidic device 120b thus exhibits viscosity dependent fluid flow resistance and in particular, a negative flowrate response to decreasing fluid viscosity.

In the depicted configuration, the first fluidic device 120a and the second fluidic device 120b are arranged in parallel in the fluid circuit defined in the flow control section 216. The first and second fluidic devices 120a,b share a common fluid source from the annular region 220, and a common fluid discharge into the central flow passage 218 via the production port(s) 204. In this configuration, the first and second fluidic devices 120a,b exhibit a common upstream pressure and a common downstream pressure. Accordingly, as the resistance to fluid flow through the fluidic devices 120a,b changes, the ratio of the flowrates through the fluidic devices 120a,b will also change. For example, as the oil to water ratio of the production fluid decreases, the viscosity of the fluid also decreases. As the viscosity of the fluid flowing through the first fluidic device 120a decreases, the resistance to flow correspondingly decreases. At the same time, as the viscosity of the fluid flowing through the second fluidic device 120b decreases, the resistance to flow correspondingly increases.

As the relative resistances change with upstream and downstream pressures being common, the relative flowrates also change. In the depicted configuration, as the oil to water ratio decreases, the ratio of the flowrate through first fluidic device 120a to the flowrate through the second fluidic device 120b increases. In other words, the flowrate through first fluidic device 120a will become progressively greater relative to the flowrate through the second fluidic device 120b due to the positive flowrate response to decreasing fluid viscosity of first fluidic device 120a and the negative flowrate response to decreasing fluid viscosity of the second fluidic device 120b. In at least one embodiment, a turbulizer or a static mixer (not shown) may be positioned upstream of one or both of the fluidic devices 120a,b to create a mixed flow.

FIG. 3B is a schematic view of another example embodiment of the flow control section 216 of FIG. 2, according to one or more additional embodiments. The flow control shroud 212 (FIG. 2) has again been removed in FIG. 3B to enable viewing of the fluid circuit provided in the flow control section 216. Similar to the embodiment of FIG. 3A, the fluidic devices are again depicted as the first fluidic device 120a and the second fluidic device 120b, where the first fluidic device 120a comprises an ICD in the form of the flow tube 302, and the second fluidic device 120b comprises an AICD in the form of a fluid diode having the vortex chamber 308, the fluid guides 310, and the centrally-located outlet 312.

Unlike the embodiment of FIG. 3A, however, the first and second fluidic devices 120a,b of FIG. 3B are arranged in series in the fluid circuit provided in the flow control section 216. The fluid flowing through the first and second fluidic devices 120a,b originates from the annular region 220 and circulates first through the second fluidic device 120b. Upon exiting the second fluidic device 120b at the outlet 312, the fluid then flows to the first fluidic device 120a, as shown by the arrows 314. The fluid then circulates through the first fluidic device 120a before being discharged into the central flow passage 218 via the production port(s) 204 following the first fluidic device 120a.

It should be noted that even though the fluidic devices 120a,b have been depicted and described in FIGS. 3A-3B as having fluid flow resistance dependent on viscosity, fluid flow resistance of the fluidic devices 120a,b may be dependent upon other fluid properties. For example, fluid flow resistance through the fluidic devices 120a,b may alternatively be dependent on fluid properties such as, but not limited to, density, fluid velocity, fluid composition, and the like, without departing from the principles of the present disclosure.

According to embodiments of the present disclosure, the fluidic devices 120a,b arranged within the flow control section 216 may be used to help estimate the water cut or alternatively the oil fraction in a producing completion (e.g., the completion string 114). As used herein, the term “water cut” refers to the ratio of water produced in an incoming fluid stream from a surrounding subterranean formation as compared to the volume of total liquids produced. Alternatively, the “water cut” could refer to the ratio of water produced in an incoming fluid stream from a surrounding subterranean formation as compared to the mass of total liquids produced. The term “water cut” could also refer to a fraction of the total flow that comprises water. As used herein, the term “oil fraction” refers to the fraction of oil contained in the total liquids produced, less the fraction corresponding to the water cut. The fluidic devices 120a,b exhibit different but known flow resistances to fluids having known fluid properties (e.g., viscosity, density, etc.). Consequently, the water cut of the fluid can be estimated by measuring one or more flow conditions (e.g., fluid pressure, flow rate, etc.) of the fluid circulating through the fluidic devices 120a,b. It will be appreciated, however, that the principles of the present disclosure may also be used to estimate the gas content in an incoming fluid stream from a surrounding subterranean formation, referred to herein as the “gas cut” of the flow.

As will be appreciated, knowing the water cut (or gas cut) in a produced fluid may prove advantageous in allowing a well operator to intelligently produce fluids by limiting the production of certain types of fluids (e.g., water), and maximizing the production of other fluids (e.g., oil). More specifically, the flow control assemblies 116 may form part of an intelligent completion having one or more interval control valves that are actuatable choke or expose the production port(s) 204. Once it is determined that the water cut in a produced stream of fluid surpasses a predetermined limit, the well operator may selectively actuate the interval control valve through a specific flow control assembly 116 to choke or cease production from that production interval. This may prove advantageous in providing more efficient production operations for the well, and may also provide information used to model the reservoir and thereby increase the ultimate recovery of the formation.

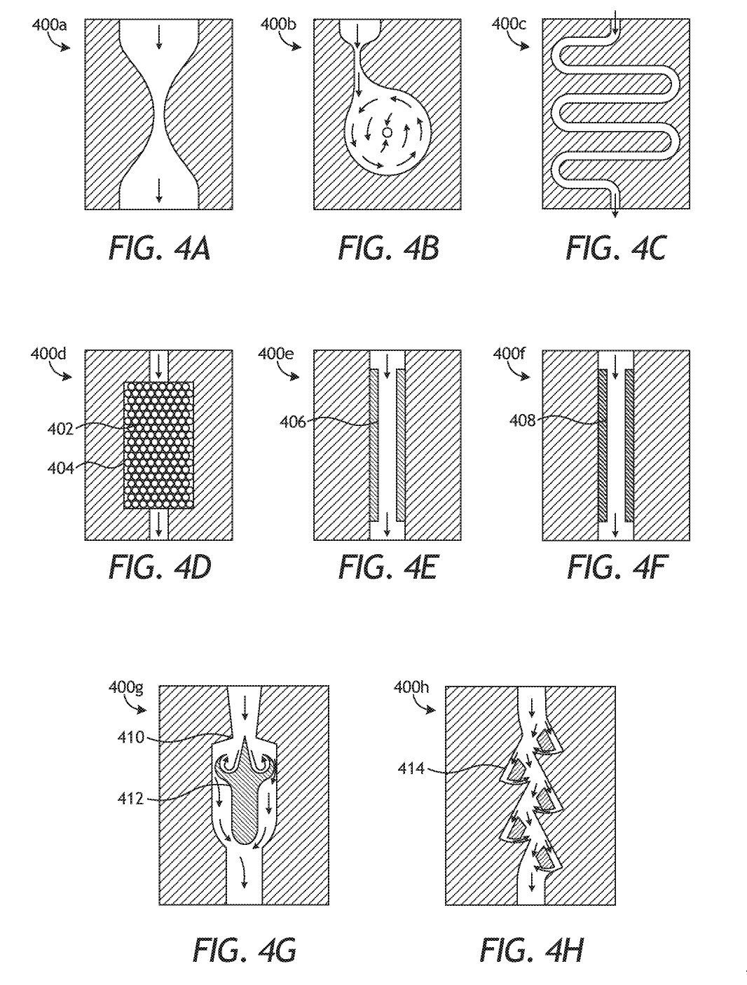

FIGS. 4A-4H are cross-sectional side views of a variety of example fluidic devices that may be employed in accordance with the principles of the present disclosure. Even though the fluidic devices 120a,b of FIGS. 3A and 3B have been depicted and described as having particular designs and configurations, the fluidic devices 120a,b used to help determine (estimate) water cut may alternatively exhibit a variety of alternate designs without departing from the scope of the present disclosure. FIGS. 4A-4H, for example, depict fluidic devices 400a through 400h, respectively, that may be employed in accordance with the principles of the present disclosure. Accordingly, the fluidic devices 120a,b of FIGS. 3A-3B may be replaced with any of the fluidic devices 400a-h.

In FIG. 4A, the fluidic device 400a is depicted generally as a nozzle. In FIG. 4B, the fluidic device 400b comprises a vortex chamber diode similar in some respects to the fluidic device 120b of FIGS. 3A-3B. In FIG. 4C, the fluidic device 400c comprises a flow tube that provides a tortuous path flow. In FIG. 4D, the fluidic device 400d comprises a porous material 402 disposed within a chamber 404. The porous material 402 may be, for example, beads or other fluid flow resisting filler materials. In FIG. 4E, the fluidic device 400e comprises a flow tube 406, similar in some respects to the fluidic device 120a of FIGS. 3A-3B. In FIG. 4F, the fluidic device 400f may include a material 408 that swells when it comes into contact with oil or water. Alternatively, the material 408 may swell in response to other stimulants such as pH, ionic concentration or the like. In FIG. 4G, the fluidic device 400g includes a converging nozzle 410 and a fluid disrupter 412 positioned downstream from the nozzle 410. In FIG. 4H, the fluidic device 400h comprises a tesla diode 414 or similar fluid diode. The fluidic devices 400a, 400c, 400d, and 400e may each be generally characterized as ICDs that have a positive flow rate response to a changing fluid property (e.g., decreasing fluid viscosity), while the fluidic devices 400b, 400g, and 400h may each be characterized as AICDs that have a negative flowrate response to the changing fluid property.

It should be noted that although the fluidic devices 400a-h are depicted as two-dimensional shapes, one or more of the fluidic devices 400a-h could exhibit a height or depth variation. For example, the vortex chamber diode of the fluidic device 400b of FIG. 4B could be conically shaped. Moreover, while not shown, one or more of the fluidic devices 400a-h may provide and otherwise include moving parts, without departing from the scope of the disclosure. Suitable fluidic devices having moving parts that may be used in accordance with the principles of the present disclosure are described in U.S. Pat. Nos. 8,875,797 and 7,823,645, and in U.S. Patent Pub. No. 2015/0040990.

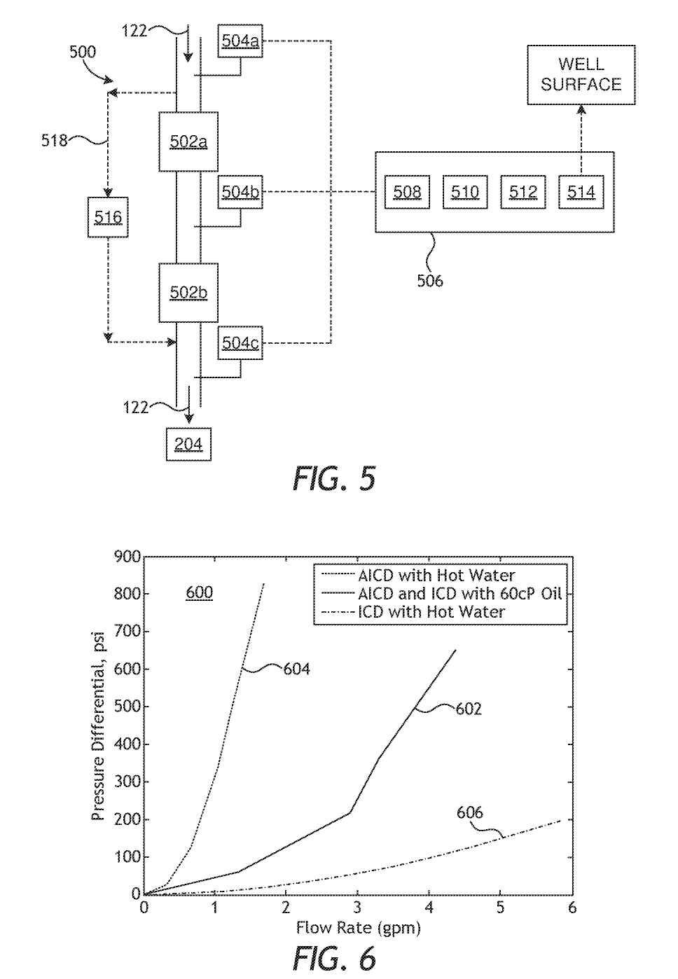

FIG. 5 is a schematic diagram of an example fluid circuit 500 used to help determine water cut (or alternatively the gas cut), according to one or more embodiments of the present disclosure. The fluid circuit 500 may be provided or otherwise defined within the flow control section 216 (FIGS. 2 and 3A-3B) of the flow control assembly 116 (FIG. 2). Accordingly, the fluid circuit 500 generally depicts the flow path for the fluid 122 originating, for example, from the subterranean formation 110 (FIG. 1), and the fluid circuit 500 may regulate the flow to the production port(s) 204 to be discharged into the central flow passage 218 (FIGS. 2 and 3A-3B). In some applications, the fluid 122 circulating through the fluid circuit 500 includes at least two fluidic constituents of water and oil. In other applications, however, the fluid 122 circulating through the fluid circuit 500 might only include a single fluidic component or phase of pure water or pure oil, for example or pure gas. In such applications, the fluid circuit 500 will nonetheless be able to measure the fluid 122 and indicate that the fluid 122 is pure.

The fluid 122 circulates through at least two fluidic devices arranged in series in the fluid circuit 500 and shown as a first fluidic device 502a and a second fluidic device 502b. The fluidic devices 502a,b may be the same as or similar to any of the fluidic devices mentioned herein, including the fluidic devices 120a,b of FIGS. 3A-3B and the fluidic devices 400a-400h of FIGS. 4A-4H. The first and second fluidic devices 502a,b, however, are different from each other and thereby exhibit different flow characteristics. In some embodiments, for instance, one may be an ICD and the other an AICD, although each may be an ICD or an AICD, without departing from the scope of the disclosure. The fluidic devices 502a,b will exhibit a different response to the flow of water, oil, and/or gas. This difference can be achieved by changes in structure, geometry, or dimensions. For example, the two fluidic devices 502a,b could both be tubes similar to the fluidic device 400e of FIG. 4E, but one may be long and skinny while the other may be short and wide.

As illustrated, the fluid circuit 500 may include a plurality of fluid sensors, shown as a first fluid sensor 504a, a second fluid sensor 504b, and a third fluid sensor 504c. The first fluid sensor 504a is communicably coupled to the fluid circuit 500 upstream of the first fluidic device 502a and configured to measure and otherwise detect a flow condition of the fluid 122 at that location. The second fluid sensor 504b is communicably coupled to the fluid circuit 500 between the first and second fluidic devices 502a,b (i.e., downstream from the first fluidic device 502a and upstream from the second fluidic device 502b), and configured to measure and otherwise detect the flow condition of the fluid 122 at that location. Lastly, the third fluid sensor 504c is communicably coupled to the fluid circuit 500 downstream of the second fluidic device 502b and configured to measure and otherwise detect the flow condition of the fluid 122 at that location.

Example flow conditions that may be measured by the sensors 504a-c include, but are not limited to fluid pressure, fluid temperature, flow rate (i.e., volumetric flow rate, mass flow rate, etc.), and fluid-induced vibrations. In some embodiments, for instance, each fluid sensor 504a-c may comprise a pressure transducer or pressure sensor configured to measure the pressure of the fluid 122 at the corresponding locations in the fluid circuit 500. In other embodiments, however, the fluid sensors 504a-c may each comprise a temperature gauge or sensor configured to monitor the temperature of the fluid 122 at the corresponding locations in the fluid circuit 500. In yet other embodiments, however, the fluid sensors 504a-c may each comprise an accelerometer or a piezoelectric component configured to monitor fluid-induced vibrations of the fluid 122 at the corresponding locations in the fluid circuit 500.

Each of the fluid sensors 504a-c may be communicably coupled (either wired or wirelessly) to a computer system 506 configured to monitor conditions in the fluid circuit 500. The computer system 506 may be located downhole, such as being included in the flow control assembly 116 (FIG. 2), or may alternatively be located at the well surface. The computer system 506 may include, for example, computer hardware and/or software used to operate the fluid sensors 504a-c. The computer hardware may include a processor 508 configured to execute one or more sequences of instructions, programming stances, or code stored on a non-transitory, computer-readable medium (e.g., a memory) and can include, for example, a general purpose microprocessor, a microcontroller, a digital signal processor, or any like suitable device.

The computer system 506 may also include a library or database 510 that stores known operational data for the fluidic devices 502a,b. Such operational data may include design and flow characteristics of each fluidic device 502a,b. As discussed below, this operational data may be accessed by the processor 508 during operation to compare the real-time data obtained by the fluid sensors 504a-c and thereby determine or otherwise estimate the water cut percentage of the fluid 122.

In some embodiments, the computer system 506 may further include a power source 512 that provides electrical power to the fluid sensors 504a-c for operation. The power source 512 may comprise, but is not limited to, one or more batteries, a fuel cell, a nuclear-based generator, a flow induced vibration power harvester, or any combination thereof.

In embodiments where the computer system 506 is located downhole, the computer system 506 may further include a bi-directional communications module 514 to enable transfer of data and/or control signals to/from the computer system 506 and a well surface location. Accordingly, the communications module 514 may be communicably coupled (either wired or wirelessly) to the well surface location to enable transfer of data or control signals to/from the well surface location during operation. The communications module 514 may include one or more transmitters and receivers, for example, to facilitate bi-directional communication with the surface location. As a result, a well operator at the well surface may be apprised of the real-time water cut percentage of the fluid circuit 500, and may be able to send command signals to the flow control assembly 116 (FIG. 2) to adjust and otherwise regulate the flow of the fluid 122 when desired.

In example operation, the fluid sensors 504a-c may each comprise pressure sensors, such as differential pressure transducers that increase the resolution of any obtained measurements. The first and second fluid sensors 504a,b detect the pressure of the fluid 122 before and after the first fluidic device 502a, respectively, and the third fluid sensor 504c detects the pressure of the fluid 122 following the second fluidic device 502b. In some embodiments, the pressure readings from the sensors 504a-c may be averaged in order to smooth the effects of potential bubble flow. More specifically, the readings from each individual sensor 504a-c could be averaged in order to reduce the sensitivity to bubble flow. Alternatively, the value calculated from the sensor readings could be averaged in order to reduce the sensitivity to bubble flow. Reducing the sensitivity to bubble flow allows for a slower processor and reduced memory requirements in the computer system 506.

Each sensor 504a-c communicates its respective readings (measurements) to the computer system 506 (located downhole or at the well surface), which calculates a pressure differential across the first and second fluidic devices 502a,b. More specifically, the computer system 506 calculates a first pressure drop (ΔP₁) across the first fluidic device 502a and a second pressure drop (ΔP₂) across the second fluidic device 502b. The computer system 506 may then calculate a pressure differential ratio (ΔP₁/ΔP₂) for the first and second fluidic devices 502a,b, and subsequently estimate the water cut of the fluid 122 based on the pressure differential ratio ΔP₁/ΔP₂.

For each fluidic device 502a,b, the amount of flow restriction depends on the average viscosity and the average density of the fluid 122 traveling therethrough and the average flow rate of the fluid 122. It is assumed that the fluid 122 includes at least two individual constituents in the form of oil and water, each of which exhibit known fluid properties, such as viscosity and density. The fluid 122 may alternatively also include an additional constituent in the form of gas. The water cut may be estimated (determined) by comparing the pressure differential ratio ΔP₁/ΔP₂ against known operational data for the first and second fluidic devices 502a,b stored in the database 510 and in view of at least one known fluid property of the fluid 122. The known fluid property of the fluid 122 may be, for example, the viscosity of the oil present in the fluid 122 or the density of the fluid 122. Such fluid properties may be generally known and obtained through well logging or sampling operations previously undertaken or during production operations.

In some embodiments, the operational data for the first and second fluidic devices 502a,b may be obtained mathematically through a numerical model for theoretical fluid flow through each fluidic device 502a,b under known conditions and using fluids having known water cuts and fluid properties. In other embodiments, the operational data for the first and second fluidic devices 502a,b may be obtained through laboratory testing of the first and second fluidic devices 502a,b. Such testing may include flowing a fluid through each fluidic device 502a,b and measuring flow conditions (e.g., pressure, temperature, flow rate, etc.) before and after each fluidic device 502a,b. The fluid will comprise a mixture of water and oil (and possibly gas) at a known water cut and the oil will exhibit a known fluid property (i.e., viscosity). This process will be repeated across a range of known water cuts and for a variety of fluid properties expected to be encountered downhole.

The operational data for the first and second fluidic devices 502a,b may then be stored in the database 510 included in the computer system 506. Upon obtaining the measured pressure differential ratio ΔP₁/ΔP₂ during downhole operation, the computer system 506 may be programmed to query the database to compare the measured pressure differential ratio ΔP₁/ΔP₂ against the known operational data for the first and second fluidic devices 502a,b and in view of the known fluid property of the fluid 122. The water cut for the fluid 122 may then be estimated based on this comparison.

FIG. 6 is a plot 600 depicting test results for two example fluidic devices that help provide operational data for the fluidic devices that might be stored in the database 510 (FIG. 5). The test results indicated in the plot 600 are obtained from a first fluidic device that comprises a fluidic device providing a fluid diode behavior (e.g., an AICD) and a second fluidic device that comprises an ICD. As will be appreciated, however, some AICDs need not exhibit or require a fluid diode behavior. The first and second fluidic devices are designed such that they each exhibit the same pressure drop when circulating oil having a viscosity of 60 centipoise (cP), as shown by the first line 602. When circulating hot water, the AICD exhibits a very large pressure differential at a small flowrate, measured in gallons per minute (gpm), as indicated by the second line 604. In contrast, when circulating hot water, the ICD exhibits a very small pressure differential at a high flowrate, as indicated by the third line 606.

As can be seen from the operational data, the AICD exhibits about sixteen times the pressure drop as compared to the ICD. Accordingly, the pressure differential ratio ΔP₁/ΔP₂ for the fluidic devices modeled in the plot 600 varies from 1:1 to 16:1, depending on the water/oil content in the fluid. For mixed flow, the pressure will vary proportionally to the oil/water fraction.

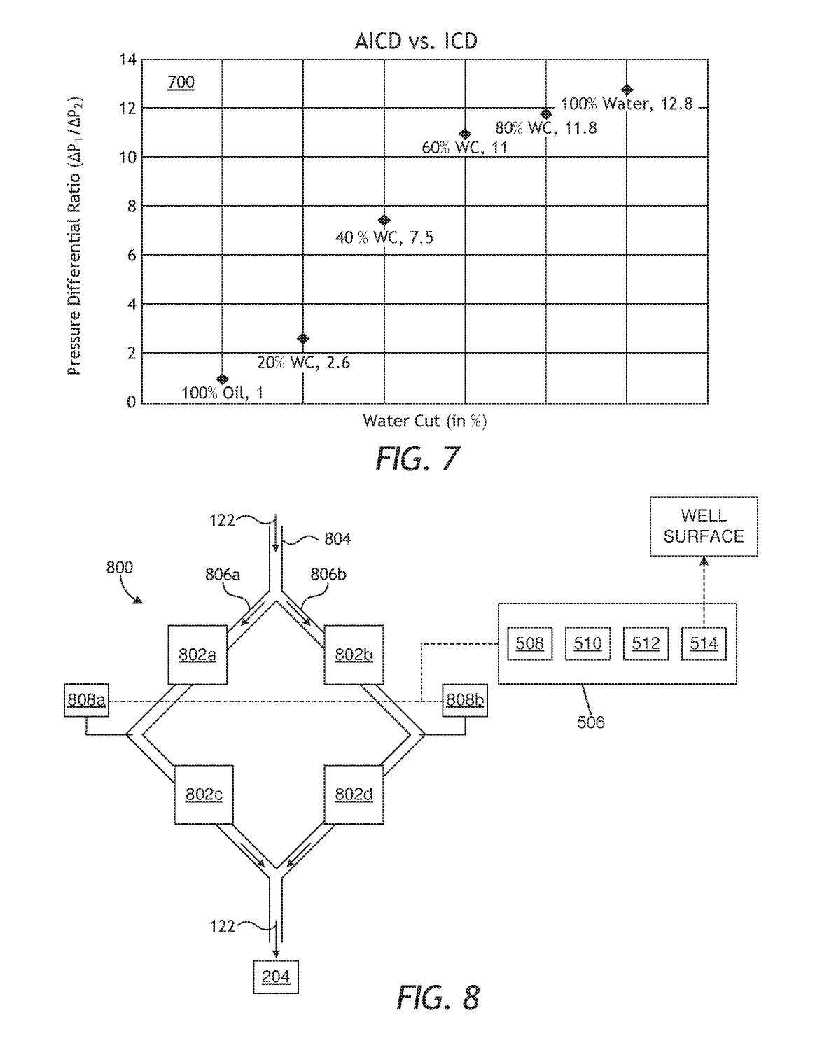

FIG. 7 is a plot 700 showing test results for the two example fluidic devices of FIG. 6 that also provide operational data for the fluidic devices that might be stored in the database 510 (FIG. 5). In the plot 700, the fluid used to obtain the operational data includes oil that exhibits a viscosity of 80 cP. By plotting the pressure differential ratio ΔP₁/ΔP₂ for the fluidic devices versus the measured water cut (WC) percentage in the fluid flow, a clear connection between the pressure ratio and the water cut can be observed. For example, when the pressure differential ratio ΔP₁/ΔP₂ is about 7.5, that may be indicative that the fluid has 40% water cut. Similarly, when the pressure differential ratio ΔP₁/ΔP₂ is about 11.8, that may be indicative that the fluid has 80% water cut.

Similar plots may be generated for fluids having different fluid properties (e.g., varying viscosity or density) or for other fluidic devices having different operational flow characteristics. The operational data provided in the plot 700, and the several other plots based on varying test parameters, may be uploaded to or stored in the database 510 of the computer system 506 (FIG. 5) and accessed by the computer system 506 to compare the real-time measured pressure differential ratio ΔP₁/ΔP₂. In view of the known fluid property of the fluid 122 (FIG. 5) circulating through the fluid circuit 500 (FIG. 5), the water cut for the fluid 122 may then be estimated based on this comparison.

Referring again to FIG. 5, in some embodiments, the computer system 506 may estimate (determine) the water cut (or alternatively the gas cut) for the fluid 122, as generally described above, and the flow rate through the fluid circuit 500 may then be estimated. More specifically, the first pressure drop ΔP₁ across the first fluidic device 502a or the second pressure drop ΔP₂ across the second fluidic device 502b may be used to estimate the flow rate through the fluid circuit 500. Once the water cut is estimated, then the first pressure drop ΔP₁ may be used to calculate the flow rate through the first fluidic device 502a or the second pressure drop ΔP₂ may be used to calculate the flow rate through the second fluidic device 502b. This is possible since the pressure drop through a fluidic device is monotonically dependent on the flow rate. Consequently, if the water cut is known, then this monotonic relationship can be used to estimate the flow rate. Accordingly, once the water cut and the fluid properties (e.g., viscosity, density, etc.) of the fluid 122 are known (or estimated), the flow rate through the fluid circuit 500 may be back-calculated based on the first or second pressure drops ΔP₁, ΔP₂.

Still referring to FIG. 5, in some embodiments, the first fluidic device 502a may be similar to or the same as the fluidic device 400d of FIG. 4D, and the second fluidic device 502b may be similar to or the same as the fluidic device 400a of FIG. 4A. In other words, the first fluidic device 502a may include a porous material disposed within a chamber and configured to provide flow resistance, while the second control device 502b may generally comprise a nozzle fluidic component. The first pressure drop ΔP₁ across the first fluidic device 502a as the fluid 122 passes through the porous media is proportional to the viscosity of the fluid 122. Moreover, the second pressure drop ΔP₂ across the second fluidic device 502b as the fluid 122 passes therethrough will be proportional to the density of the fluid 122. For oil and water with similar densities, this allows the pressure differential ratio ΔP₁/ΔP₂ to be proportional to a ratio of the viscosity.

In some embodiments, the total pressure drop across the first and second fluidic devices 502a,b may be small, such as in applications where an interval control valve or the like is used to choke or stop the flow of the fluid 122 passing through the production port(s) 204. In such applications, the pressure differential ratio ΔP₁/ΔP₂ will correspondingly be small and subject to error. Consequently, it may be advantageous to artificially increase the pressure within the fluid circuit 500 to facilitate more robust pressure readings from the fluid sensors 504a-c. To accomplish this, the fluid circuit 500 may include a restriction 516 that runs parallel to the first and second fluidic devices 502a,b. More specifically, the restriction 516 may be positioned in a bypass conduit 518 extending from upstream of the first fluidic device 502a and to downstream of the second fluidic device 502b. A portion of the fluid 122 will circulate into the bypass conduit 518 and provide an increase in back pressure upstream from the restriction, which will increase the pressure drop across the first and second fluidic devices 502a,b. In other embodiments, the restriction 516 might be positioned in the flow path running through the interval control valve or the flow path running along a drainage layer beneath the filter medium 206 (FIG. 2). As a result, the pressure differential ratio ΔP₁/ΔP₂ through the first and second fluidic devices 502a,b will be consistent even at low flow rates or at low pressure drops.

FIG. 8 is a schematic diagram of another example fluid circuit 800 used to help determine water cut (or alternatively the gas cut), according to one or more embodiments of the present disclosure. The fluid circuit 800 may be similar in some respects to the fluid circuit 500 of FIG. 5, such as being provided or otherwise defined within the flow control section 216 (FIGS. 2 and 3A-3B) of the flow control assembly 116 (FIG. 2) to provide a flow path for the fluid 122. Unlike the fluid circuit 500 of FIG. 5, however, the fluid circuit 800 may prove advantageous where the measured pressure drops are small.

As illustrated, the fluid 122 circulates through at least four fluidic devices shown as a first fluidic device 802a, a second fluidic device 802b, a third fluidic device 802c, and a fourth fluidic device 802d. The fluidic devices 802a-d may be the same as or similar to any of the fluidic devices mentioned herein, including the fluidic devices 120a,b of FIGS. 3A-3B and the fluidic devices 400a-400h of FIGS. 4A-4H. In some embodiments, the first and fourth fluidic devices 802a,d are the same type of fluidic diode and the second and third fluidic devices 802b,c are the same type of fluidic diode. Moreover, in such embodiments, the first and fourth fluidic devices 802a,d are different from the second and third fluidic devices 802b,c and thereby exhibit different flow characteristics.

The fluid circuit 800 includes a main fluid conduit 804 that splits into a first branch conduit 806a and a second branch conduit 806b that eventually meet up again (i.e., fluidly communicate) downstream. The first and third fluidic devices 802a,c are arranged in the first branch conduit 806a, and the second and fourth fluidic devices 802b,d are arranged in the second branch conduit 806b. Accordingly, the fluid circuit 800 may be arranged similar to a Wheatstone bridge configuration or an H-bridge configuration.

As illustrated, the fluid circuit 800 may also include at least two fluid sensors, shown as a first fluid sensor 808a and a second fluid sensor 808b. The first fluid sensor 808a is communicably coupled to the fluid circuit 800 in the first branch conduit 806a downstream from the first fluidic device 802a but upstream from the fourth fluidic device 802d and configured to measure a flow condition of the fluid 122 at that location. The second fluid sensor 808b is communicably coupled to the fluid circuit 800 in the second branch conduit 806b downstream from the second fluidic device 802b but upstream from the third fluidic device 802c and configured to measure and otherwise detect the flow condition of the fluid 122 at that location.

If the pressure drop across the first and second fluidic devices 802a,b is small, then the effect can be amplified by measuring the pressure at the first and second fluid sensors 808a,b and communicating those measurements to the computer system 506 for processing. The computer system 506 may then calculate the pressure differential between the first and second fluid sensors 808a,b. As will be appreciated, this may allow for less uncertainty in measuring the pressure differential ratio ΔP₁/ΔP₂ in estimating the water cut of the fluid 122, as generally described above. Alternatively, in some embodiments, the differential pressure at the first and second fluid sensors 808a,b may be measured to provide a qualitative estimate for the water cut. The differential pressure could be measured, for example, with a diaphragm.

Measuring the pressure at the first and second fluid sensors 808a,b may also allow for estimating three-phase flow fractions. Three-phase flow fractions can be achieved by using different fluidic devices with known flow characteristics for each fluidic device 802a-d. The processor 508 may be configured or otherwise programmed to obtain three measurements, the pressure at the location of the first fluid sensor 808a, the pressure at the location of the second fluid sensor 808b, and the differential pressure between the first and second fluid sensors 808a,b. With these three measurements, the processor 508 may be programmed to estimate the three phases of the fluid flow.

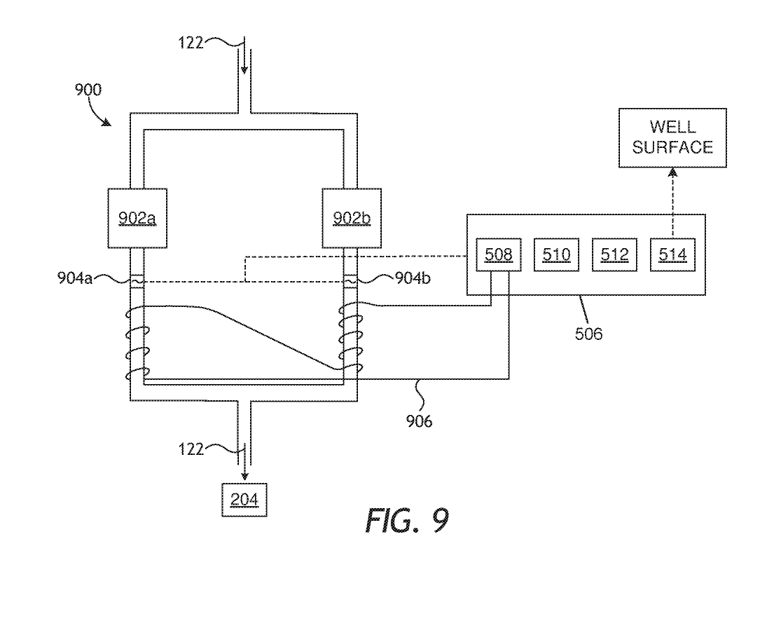

FIG. 9 is a schematic diagram of another example fluid circuit 900 used to help estimate water cut (or alternatively the gas cut), according to one or more embodiments of the present disclosure. The fluid circuit 900 may be similar in some respects to the fluid circuit 500 of FIG. 5 and, therefore, may be provided or otherwise defined within the flow control section 216 (FIGS. 2 and 3A-3B) of the flow control assembly 116 (FIG. 2), and provides a flow path for the fluid 122. Unlike the fluid circuit 500 two fluidic devices are arranged in parallel in the fluid circuit 900 and shown as a first fluidic device 902a and a second fluidic 902b. The fluidic devices 902a,b may be the same as or similar to any of the fluidic devices mentioned herein, including the fluidic devices 120a,b of FIGS. 3A-3B and the fluidic devices 400a-400h of FIGS. 4A-4H. The first and second fluidic devices 902a,b, however, are different from each other and thereby exhibit different flow characteristics. For instance, one may be an ICD and the other an AICD, although each may be an ICD or an AICD, without departing from the scope of the disclosure.

While only two fluidic devices 902a,b are shown in FIG. 9, it will be appreciated that more than two may be used, without departing from the scope of the disclosure. Additional fluid devices may be employed and advantageous in helping to reduce the uncertainty of the measurement as well as to help identify if there are more than two phases present in the fluid 122.

With the first and second fluidic devices 902a,b in fluid parallel, the same pressure drop will be experienced across each fluidic device 902a,b. Accordingly, it may be necessary to measure another flow condition besides fluid pressure in estimating the water cut of the fluid 122. In the illustrated embodiment, the fluid circuit 900 further includes a first fluid sensor 904a arranged downstream from the first fluidic device 902a, and a second fluid sensor 904b arranged downstream from the second fluidic device 902b. The fluid sensors 904a,b are configured to measure the flow rate (i.e., volumetric flow rate, mass flow rate, etc.) through the first and second fluidic devices 902a,b, respectively. Knowing the flow rate of the fluid 122 in conjunction with known operational data of the first and second fluidic devices 902a,b may prove useful in estimating the water cut of the fluid 122.

The fluid sensors 904a,b may comprise any sensor, gauge, or means capable of determining flow rate through a fluid conduit. In some embodiments, for instance, the fluid sensors 904a,b may each comprise a flow meter, but could alternatively comprise distributed acoustic sensors (DAS), distributed temperature sensors (DTS), or any other known sensors or flow-measuring means.

In at least one embodiment, the first fluidic device 902a may comprise a nozzle, similar to the fluidic device 400e of FIG. 4E, and the second fluidic device 902b may comprise a vortex chamber diode, similar to the fluidic device 120b of FIGS. 3A-3B or the fluidic device 400b of FIG. 4B. In such embodiments, the first and second fluid sensors 904a,b may each comprise an electronic flow meter or the like and report their corresponding measurements to the computer system 506 for processing. The first fluid sensor 904a may be configured to measure and report a first mass flow rate (m₁) or a first fluid velocity (Q₁), while the second fluid sensor 904b may be configured to measure and report a second mass flow rate (m₂) or a second fluid velocity (Q₂).

The computer system 506 may access the operational data for the first and second fluid sensors 904a,b from the database 510 in conjunction with the known fluid properties of the fluid 122. If the fluid 122 has a higher proportion of water (i.e., a higher water cut), for example, then the first fluid sensor 904a will return a reading greater than the second fluid sensor 904b as more water will pass through the first fluidic device 902a as compared to the second fluidic device 902b. In contrast, if the fluid 122 has a higher proportion of oil (i.e., a lower water cut), then the first fluid sensor 904a will return a reading lower than the second fluid sensor 904b as more oil will pass through the second fluidic device 902b as compared to the first fluidic device 902a. The processor 508 may be configured to calculate the ratio of the mass flow rates (m₁/m₂) or the ratio of the fluid velocity (Q₁/Q₂) and use these ratios in a manner analogous to how the measured pressure differential ratio ΔP₁/ΔP₂ of FIG. 5 above was used in calculating water cut. Accordingly, the ratio of the mass flow rates (m₁/m₂) or the ratio of the fluid velocity (Q₁/Q₂) can be used to quantitatively estimate the water cut fraction.

In at least one embodiment, the first fluidic device 902a may comprise a nozzle, similar to the fluidic device 400e of FIG. 4E, and the second fluidic device 902b may comprise long tube, similar to the fluidic device 400c of FIG. 4C. In such embodiments, the first and second fluid sensors 904a,b may each comprise a vortex flow meter, and a fiber optic cable 906 may be used to sense acoustic or temperature fluctuations following the first and second fluid sensors 904a,b. In some applications, for instance, the bluff body in each vortex flow meter will shed Karman vortices, and the vortex shedding frequency is proportional to the flow velocity. In such applications, the fiber optic cable 906 may operate as a distributed acoustic sensor (DAS) by sensing the vibrations (fluid fluctuations) emanating from each vortex flow meter and generated by the Karman vortices. In other embodiments, however, the fiber optic cable 906 may operate as a distributed temperature sensor (DAS) by sensing the Joule-Thomson heating emanating from each vortex flow meter during operation. Measurements obtained by the first and second fluid sensors 904a,b and the fiber optic cable 906 may be transmitted to the computer system 506 for processing in estimating the water cut.

Embodiments disclosed herein include:

A. A method that includes drawing a fluid into a flow control assembly coupled to a completion string positioned within a wellbore, the flow control assembly including a first fluidic device and a second fluidic device, where the first and second fluidic devices exhibit different flow characteristics, measuring a flow condition of the fluid circulating through the first and second fluidic devices with a plurality of fluid sensors, and estimating a water cut of the fluid based on the flow condition measured by the plurality of fluid sensors.

B. A completion string that includes a base pipe that defines a central flow passage and one or more flow ports, a flow control assembly coupled to the base pipe and including a first fluidic device and a second fluidic device, where the first and second fluidic devices exhibit different flow characteristics, a plurality of fluid sensors that measure a flow condition of a fluid circulating through the first and second fluidic devices, and a computer system communicably coupled to the plurality of fluid sensors and programmed to estimate a water cut of the fluid based on the flow condition measured by the plurality of fluid sensors.

Each of embodiments A and B may have one or more of the following additional elements in any combination: Element 1: wherein the first fluidic device exhibits a positive flowrate response to decreasing fluid viscosity, and the second fluidic device exhibits a negative flowrate response to decreasing fluid viscosity. Element 2: wherein the first and second fluidic devices are arranged in series and measuring the flow condition of the fluid comprises measuring the flow condition upstream of the first fluidic device with a first fluid sensor of the plurality of fluid sensors, measuring the flow condition downstream of the first fluidic device with a second fluid sensor of the plurality of fluid sensors, and measuring the flow condition downstream of the second fluidic device with a third fluid sensor of the plurality of fluid sensors. Element 3: wherein the flow condition comprises fluid pressure and estimating the water cut of the fluid comprises calculating a first pressure drop across the first fluidic device based on measurements obtained from the first and second fluid sensors, calculating a second pressure drop across the second fluidic device based on measurements obtained from the second and third fluid sensors, calculating a pressure differential ratio between the first and second fluidic devices, and estimating the water cut of the fluid based on the pressure differential ratio. Element 4: further comprising averaging the measurements obtained from each of the first, second, and third fluid sensors to smooth effects of potential bubble flow in the fluid. Element 5: wherein estimating the water cut of the fluid based on the pressure differential ratio comprises comparing the pressure differential ratio against known operational data for the first and second fluidic devices and further against a known fluid property of the fluid. Element 6: further comprising estimating a flow rate of the fluid through the first and second fluidic devices based on the first pressure drop or the second pressure drop. Element 7: further comprising conveying a portion of the fluid through a bypass conduit in parallel with the first and second fluidic devices, and increasing the fluid pressure with a restriction positioned in the bypass conduit. Element 8: wherein the first and second fluidic devices are arranged in parallel and the flow condition is a flow rate of the fluid, the method further comprising measuring the flow rate of the fluid downstream of the first fluidic device with a first fluid sensor of the plurality of fluid sensors and thereby obtaining a first mass flow rate or fluid velocity, measuring the flow rate of the fluid downstream of the second fluidic device with a second fluid sensor of the plurality of fluid sensors and thereby obtaining a second mass flow rate or fluid velocity, and estimating the water cut of the fluid based on the first and second mass flow rates or fluid velocities and known flow characteristics of the first and second fluidic devices. Element 9: wherein the first and second fluid sensors are vortex flow meters, the method further comprising sensing acoustic or temperature fluctuations downstream from the first fluid sensor with a fiber optic cable, sensing acoustic or temperature fluctuations downstream from the second fluid sensor with the fiber optic cable, and estimating the water cut of the fluid based on the first and second flow rates and measurements obtained by the fiber optic cable. Element 10: further comprising altering a flow of the fluid based on the water cut. Element 11: further comprising estimating a gas cut of the fluid based on the flow condition measured by the plurality of fluid sensors.

Element 12: wherein the first fluidic device exhibits a positive flowrate response to decreasing fluid viscosity, and the second fluidic device exhibits a negative flowrate response to decreasing fluid viscosity. Element 13: wherein the first fluidic device comprises a flow tube and the second fluidic device comprises a vortex chamber diode. Element 14: wherein the first and second fluidic devices are arranged in series and the plurality of fluid sensors comprises a first fluid sensor that measures the flow condition upstream of the first fluidic device, a second fluid sensor that measures the flow condition downstream of the first fluidic device, and a third fluid sensor that measures the flow condition downstream of the second fluidic device. Element 15: wherein the flow condition comprises fluid pressure and the computer system is programmed to calculate a first pressure drop across the first fluidic device based on measurements obtained from the first and second fluid sensors, calculate a second pressure drop across the second fluidic device based on measurements obtained from the second and third fluid sensors, calculate a pressure differential ratio between the first and second fluidic devices, and estimate the water cut of the fluid based on the pressure differential ratio. Element 16: wherein the computer system includes a database that stores known operational data for the first and second fluidic devices, and wherein the computer system is further programmed to compare the pressure differential ratio against the known operational data and further against a known fluid property of the fluid. Element 17: wherein the first and second fluidic devices are arranged in parallel and the flow condition is a flow rate of the fluid, and wherein the plurality of fluid sensors comprises a first fluid sensor that measures the flow rate of the fluid downstream of the first fluidic device and thereby obtains a first mass flow rate or fluid velocity, and a second fluid sensor that measures the flow rate of the fluid downstream of the second fluidic device and thereby obtains a second mass flow rate or fluid velocity, and wherein the water cut of the fluid is estimated based on the first and second mass flow rates or fluid velocities and known flow characteristics of the first and second fluidic devices. Element 18: wherein the first and second fluid sensors are vortex flow meters, the flow control assembly further comprising a fiber optic cable that senses acoustic or temperature fluctuations downstream from the first fluid sensor and the second fluid sensor, wherein the water cut of the fluid is estimated based on the first and second flow rates and measurements obtained by the fiber optic cable. Element 19: wherein the computer system includes a bi-directional communications module that enables communication between the flow control assembly and a well surface location.

By way of non-limiting example, exemplary combinations applicable to A and B include: Element 2 with Element 3; Element 3 with Element 4; Element 3 with Element 4; Element 5 with Element 6; Element 5 with Element 7; Element 8 with Element 9; Element 14 with Element 15; Element 15 with Element 16; and Element 17 with Element 18.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

1. A method, comprising:
drawing a fluid into a flow control assembly coupled to a completion string positioned within a wellbore, the flow control assembly including a first fluidic device and a second fluidic device, where the first and second fluidic devices exhibit different flow characteristics;
measuring a flow condition of the fluid circulating through the first and second fluidic devices with a plurality of fluid sensors; and
estimating a water cut of the fluid based on the flow condition measured by the plurality of fluid sensors.

2. The method of claim 1, wherein the first fluidic device exhibits a positive flowrate response to decreasing fluid viscosity, and the second fluidic device exhibits a negative flowrate response to decreasing fluid viscosity.

3. The method of claim 1, wherein the first and second fluidic devices are arranged in series and measuring the flow condition of the fluid comprises:
measuring the flow condition upstream of the first fluidic device with a first fluid sensor of the plurality of fluid sensors;
measuring the flow condition downstream of the first fluidic device with a second fluid sensor of the plurality of fluid sensors; and
measuring the flow condition downstream of the second fluidic device with a third fluid sensor of the plurality of fluid sensors.

4. The method of claim 3, wherein the flow condition comprises fluid pressure and estimating the water cut of the fluid comprises:
calculating a first pressure drop across the first fluidic device based on measurements obtained from the first and second fluid sensors;
calculating a second pressure drop across the second fluidic device based on measurements obtained from the second and third fluid sensors;
calculating a pressure differential ratio between the first and second fluidic devices; and
estimating the water cut of the fluid based on the pressure differential ratio.

5. The method of claim 4, further comprising averaging the measurements obtained from each of the first, second, and third fluid sensors to smooth effects of potential bubble flow in the fluid.

6. The method of claim 4, wherein estimating the water cut of the fluid based on the pressure differential ratio comprises comparing the pressure differential ratio against known operational data for the first and second fluidic devices and further against a known fluid property of the fluid.

7. The method of claim 6, further comprising estimating a flow rate of the fluid through the first and second fluidic devices based on the first pressure drop or the second pressure drop.

8. The method of claim 6, further comprising:
conveying a portion of the fluid through a bypass conduit in parallel with the first and second fluidic devices; and
increasing the fluid pressure with a restriction positioned in the bypass conduit.

9. The method of claim 1, wherein the first and second fluidic devices are arranged in parallel and the flow condition is a flow rate of the fluid, the method further comprising:
measuring the flow rate of the fluid downstream of the first fluidic device with a first fluid sensor of the plurality of fluid sensors and thereby obtaining a first mass flow rate or fluid velocity;
measuring the flow rate of the fluid downstream of the second fluidic device with a second fluid sensor of the plurality of fluid sensors and thereby obtaining a second mass flow rate or fluid velocity; and
estimating the water cut of the fluid based on the first and second mass flow rates or fluid velocities and known flow characteristics of the first and second fluidic devices.

10. The method of claim 9, wherein the first and second fluid sensors are vortex flow meters, the method further comprising:
sensing acoustic or temperature fluctuations downstream from the first fluid sensor with a fiber optic cable;
sensing acoustic or temperature fluctuations downstream from the second fluid sensor with the fiber optic cable; and
estimating the water cut of the fluid based on the first and second flow rates and measurements obtained by the fiber optic cable.

11. The method of claim 1, further comprising altering a flow of the fluid based on the water cut.

12. The method of claim 1, further comprising estimating a gas cut of the fluid based on the flow condition measured by the plurality of fluid sensors.

13. A completion string, comprising:
a base pipe that defines a central flow passage and one or more flow ports;
a flow control assembly coupled to the base pipe and including a first fluidic device and a second fluidic device, where the first and second fluidic devices exhibit different flow characteristics;
a plurality of fluid sensors that measure a flow condition of a fluid circulating through the first and second fluidic devices; and
a computer system communicably coupled to the plurality of fluid sensors and programmed to estimate a water cut of the fluid based on the flow condition measured by the plurality of fluid sensors.

14. The completion string of claim 13, wherein the first fluidic device exhibits a positive flowrate response to decreasing fluid viscosity, and the second fluidic device exhibits a negative flowrate response to decreasing fluid viscosity.

15. The completion string of claim 13, wherein the first fluidic device comprises a flow tube and the second fluidic device comprises a vortex chamber diode.

16. The completion string of claim 13, wherein the first and second fluidic devices are arranged in series and the plurality of fluid sensors comprises:
a first fluid sensor that measures the flow condition upstream of the first fluidic device;
a second fluid sensor that measures the flow condition downstream of the first fluidic device; and
a third fluid sensor that measures the flow condition downstream of the second fluidic device.

17. The completion string of claim 16, wherein the flow condition comprises fluid pressure and the computer system is programmed to:
calculate a first pressure drop across the first fluidic device based on measurements obtained from the first and second fluid sensors;
calculate a second pressure drop across the second fluidic device based on measurements obtained from the second and third fluid sensors;
calculate a pressure differential ratio between the first and second fluidic devices; and
estimate the water cut of the fluid based on the pressure differential ratio.

18. The completion string of claim 17, wherein the computer system includes a database that stores known operational data for the first and second fluidic devices, and wherein the computer system is further programmed to compare the pressure differential ratio against the known operational data and further against a known fluid property of the fluid.

19. The completion string of claim 13, wherein the first and second fluidic devices are arranged in parallel and the flow condition is a flow rate of the fluid, and wherein the plurality of fluid sensors comprises:
a first fluid sensor that measures the flow rate of the fluid downstream of the first fluidic device and thereby obtains a first mass flow rate or fluid velocity; and
a second fluid sensor that measures the flow rate of the fluid downstream of the second fluidic device and thereby obtains a second mass flow rate or fluid velocity, and wherein the water cut of the fluid is estimated based on the first and second mass flow rates or fluid velocities and known flow characteristics of the first and second fluidic devices.

20. The completion string of claim 19, wherein the first and second fluid sensors are vortex flow meters, the flow control assembly further comprising:
a fiber optic cable that senses acoustic or temperature fluctuations downstream from the first fluid sensor and the second fluid sensor,
wherein the water cut of the fluid is estimated based on the first and second flow rates and measurements obtained by the fiber optic cable.

21. (canceled)
